(19) United States
(12) Patent Application Publication
(10) Pub. No.: US 2022/0226230 A1
(43) Pub. Date: Jul. 21, 2022

(54) Composition, Method of Making, Method of Use and System for Hair Health

(71) Applicant: BLEUVERSE LLC, Wylie, TX (US)

(72) Inventor: Andy NGUYEN, Wylie, TX (US)

(21) Appl. No.: 17/151,093

(22) Filed: Jan. 15, 2021

(51) Int. Cl. A61K 8/92; A61K 8/9789; A61Q 5/00; A61K 8/04; A61K 8/9794

(52) U.S. Cl. CPC A61K 8/922; A61K 8/9789; A61K 8/9794; A61K 8/04; A61Q 5/00

(57) ABSTRACT

Composition to increase the thickness of human hair, system including the composition, method of making the composition and method of using the composition. The composition comprises essential oil and carrier oil that is miscible with the essential oil. The essential oil comprises lemongrass oil, helichrysum oil, lavender oil, clary sage oil, cedarwood oil, eucalyptus oil, thyme oil , bergamot oil, tea tree oil, rosemary oil, and peppermint oil. The system comprises the composition, a container, and instructions for use of the composition. The composition is located inside the container and contained by the container. The instructions comprise an instruction to apply the composition to a human scalp. The method of making the composition comprises providing the carrier oil and adding the essential oil to the carrier oil. The method of using the composition comprises applying the composition to a human scalp.

### CROSS-REFERENCE TO RELATED APPLICATIONS

None.

### BACKGROUND

### Technical Field

The present invention relates to applications to enhance the health, restoration and growth of hair and to mitigate, stop or reverse hair loss.

### Background

Existing products for hair health, restoration, and growth (i) lack a desired degree of effectiveness, (ii) use formulations with components and chemical that consumers would prefer to avoid, (iii) do not use components that are organic or natural, (iv) do not employ essential oils, in part or entirely, or (iv) are not satisfactory for any combination of items (i)-(iv).

### SUMMARY OF THE INVENTION

In one aspect, the present disclosure provides a composition to increase the thickness of human hair, the composition comprising essential oil and carrier oil that is miscible with the essential oil in any mass ratio. The essential oil comprises lemongrass oil, helichrysum oil, lavender oil, clary sage oil, cedarwood oil, eucalyptus oil, thyme oil , bergamot oil, tea tree oil, rosemary oil, and peppermint oil.

In a second aspect, the present disclosure provides a method of making the composition, the method comprising several steps. A first step comprises providing the carrier oil. A second step comprises adding the essential oil to the carrier oil.

In a third aspect, the present disclosure provides a method of using the composition, the method comprising applying the composition to a human scalp.

In a fourth aspect, the present disclosure provides a system comprising the composition, a container, and instructions for use of the composition. The composition is located inside the container and contained by the container. The instructions comprise an instruction to apply the composition to a human scalp.

Other aspects, embodiments and features of the invention will become apparent from the following detailed description of the invention when considered in conjunction with the accompanying drawings. The accompanying figures are schematic and are not intended to be drawn to scale. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a flow chart illustrating a method for making a composition according to this disclosure.

FIG. 2 is a flow chart illustrating a method for using a composition according to this disclosure.

FIG. 3 is an image of a male human head taken on Apr. 23, 2019.

FIG. 4 is an image of a male human head taken on Oct. 22, 2020, after the application of compositions in accordance with this disclosure.

FIG. 5 depicts an illustrative example of a lid separated from the body of a container for a composition of this disclosure.

FIG. 6 depicts the lid secured to the body of the container illustrated in FIG. 5.

### DETAILED DESCRIPTION

This disclosure describes compositions that can be topically applied to a human scalp. The disclosure also describes methods of making the compositions and methods of using the compositions.

The compositions can be used to support healthy hair, to enhance the health of hair, restore the health of hair follicles that have become less healthy or shrunken, and mitigate, stop or reverse hair loss. Advantageously, the composition can provide one or more of these benefits without components and chemical that some consumers would prefer to avoid. In some embodiments, the components of the composition can substantially or completely derived from natural sources, organic, or both. In some embodiments, the composition is substantially or completely made up of carrier oils and essential oils.

An embodiment of a composition in accordance with this disclosure will now be described with reference to Table 1.

TABLE 1
Illustrative Composition

| COMPONENT OF | VOLUME (mL) | VOLUME % (rounded to |
|---|---|---|
| COMPOSITION | IN COMPOSITION | 2 decimal places) |
| Extra Virgin Coconut Oil | 100 | 16.34 |
| Apricot Oil (e.g., Apricot | 100 | 16.34 |
| Kernel Oil), Castor Oil or | | |
| any combination thereof | | |
| Grapeseed Oil | 100 | 16.34 |
| Sweet Almond Oil | 100 | 16.34 |
| Virgin Avocado Oil | 100 | 16.34 |
| Jojoba Oil | 100 | 16.34 |
| Carrier Oil Subtotal | 600 | 98.04 |
| Lemongrass Oil | 1.1 | 0.18 |
| Helichrysum Oil | 1.1 | 0.18 |
| Lavender Oil | 1.1 | 0.18 |
| Clary Sage Oil | 1.1 | 0.18 |
| Cedarwood Oil | 1.1 | 0.18 |
| Eucalyptus Oil | 1.1 | 0.18 |
| Thyme Oil | 1.1 | 0.18 |
| Bergamot Oil | 1.1 | 0.18 |
| Tea Tree Oil | 1.1 | 0.18 |
| Rosemary Oil | 1.1 | 0.18 |
| Peppermint Oil | 1.1 | 0.18 |
| Essential Oil Subtotal | 12.1 | 1.98 |
| COMPOSITION TOTAL | 612.1 | 100.02 |

As can be seen, the composition of Table 1 comprises two subcomponents: a carrier oil subcomponent, and an essential oil subcomponent. The carrier oil makes up approximately 98% by volume of the composition, and the essential oil makes up approximately 2% by volume of the composition.

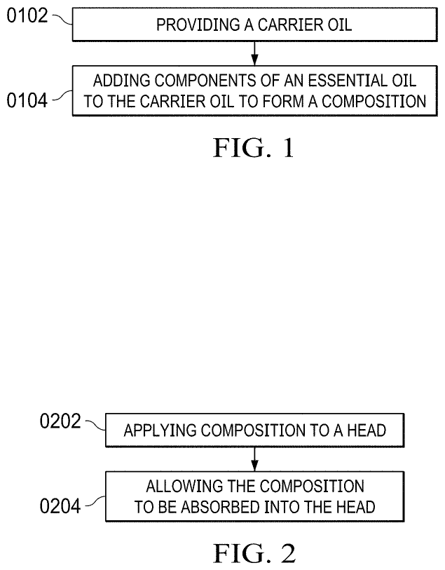

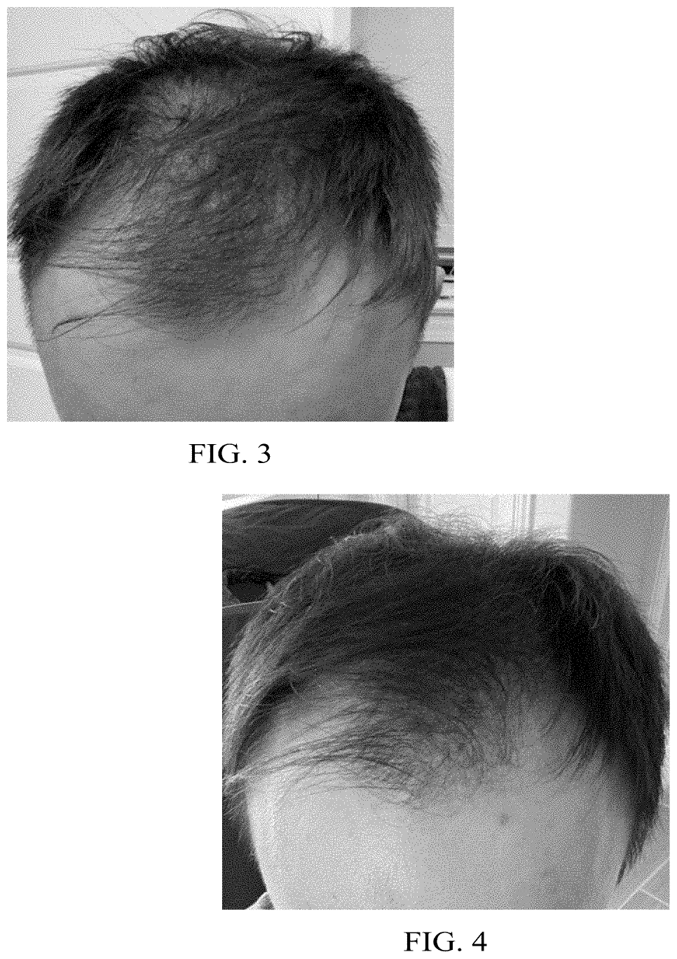

An embodiment of a method for making a composition as described in this disclosure will now be described with reference to FIG. 1 and Table 1. As can be seen, a first step of the method is providing 0102 a carrier oil. With reference to Table 1, the carrier oil can comprise one, a selection of, or all of the listed essential oil components. Including multiple essential oils can be useful for several reasons. First, by using multiple essential oils at lower concentrations, the risk of an allergic reaction can be reduced. Second, if one essential oil, for example, grape see oil, is preferred but more costly, a composition can include this preferred oil along with other oils. This can help to provide some of the desirable attributes of the preferred carrier oil, for example, miscibility with the essential oils, less smell, less stickiness, less viscosity, more optical clarity, or any combination thereof, while also helping to reduce the overall cost of the composition. Importantly, when coconut oil, or any other oil with a melting point near or above room temperature, is a carrier oil, it can be helpful to add the coconut oil or other oil to at least one other component of the carrier oil in liquid form. This can require heating to melt the coconut oil or other oil before adding the coconut oil to at least one other component of the carrier oil. Advantageously, after mixing the coconut oil or other oil with the at least one other component of the carrier oil to form a mixture, the coconut oil or other oil can remain as a liquid, even if the temperature of the mixture drops below the melting temperature of the coconut oil or other oil. This can be useful as other components are added to the mixture to form the carrier oil. This can also be useful as components of the essential oil are added to the carrier oil.

With reference again to FIG. 1, the method of making the composition also comprises adding 0104 components of an essential oil to the carrier oil to form the composition. It can be advantageous to stir the carrier oil, for example, with an overhead mechanical stirrer, while the components of the essential oil are being added. It can also be useful to add one component of the essential oil to the carrier oil at a time. Although, in some embodiments, a plurality of essential oils that are not mutually reactive or not significantly mutually reactive can be added to the carrier oil at the same time. After adding each component or combination of components of the essential oil to the carrier oil, it can be advantageous to stir the resulting mixture for a sufficient time (e.g., 3 to 5 minutes) to achieve a homogenous mixture or substantially homogeneous mixture before adding any new essential oil components to the resulting mixture. It can also be useful to add each essential oil to the carrier oil using a dedicated delivery and measurement system (e.g., measuring cup, etc.) or at least a delivery and measurement system that has been cleaned if it was previously used with another essential oil. Furthermore, in some embodiments, it can be helpful to add the essential oils in the order listed in Table 1. Furthermore, even if additional essential oils are included in the composition relative to the essential oils listed in Table 1, or if fewer than all the essential oils listed in Table 1 are included in the composition, it can be useful to add the components of the essential oil to the carrier oil in the order listed in Table 1.

With reference now to FIG. 2, a method of using the composition will be disclosed. A first step of the method comprises applying 0202 the composition to a head. A second step comprises allowing 0204 the composition to be absorbed into the head. In some embodiments, it can be useful to shake the composition well before use to provide a homogenous composition or to at least increase the homogeneity of the composition.

In some embodiments, it can be useful to permit the composition to be absorbed into the head for at least two hours before the composition is washed off. In some embodiments, it is recommended that the composition be applied at least three times a week. For example, the composition can be applied every other day or every two days. In some embodiments, the step of applying the composition to the head comprises applying the composition to the head so that there are 1 to 2 intervening non-application days separating consecutive application days. The intervening non-application days are days when the composition is not applied to the head. The consecutive application days are days when the composition is applied to the head. To illustrate, given a series of 8 consecutive days, if the composition is applied on Monday, not applied on Tuesday, not applied on Wednesday, applied on Thursday, not applied on Friday, applied on Saturday, not applied on Sunday, and applied on the immediately following Monday, then the first Monday and Thursday are consecutive application days and Tuesday and Wednesday are intervening non-application days. Similarly, Thursday and Saturday are consecutive application days and Friday is an intervening non-application day. Likewise, Saturday and the immediately following Monday are consecutive application days and Sunday is an intervening non-application day.

In some embodiments, the composition can be gently massaged into the scalp, for example, with fingers or fingertips. This can be effective to help ensure that the composition reaches the roots of the hair in the scalp.

In some embodiments, the composition is applied to the scalp (e.g., the portion of the head that is or was previously covered with hair). In some embodiments, the composition is applied only to the top of the scalp.

In some embodiments, the amount of the composition applied to the human scalp is about 1 to 5 mL, 2 to 4 mL, 1.5 to 2.5 mL, 2 mL or 3 mL. In some embodiments, the amount of the composition applied to a head is selected to provide about 1 mL of the composition per 55 to 150 cm\hat{ }2 of surface area of scalp to which the composition is applied, which is equivalent to about 0.018 to 0.0067 mL/cm\hat{ }2. For a typical person having a head circumference of about 53 to 59 cm for a female or about 56 to 61 cm for a male, the area of the top of the head to which the composition is applied can be in the range of about 223 to 277 cm\hat{ }2 for a female and about 249 to 296 cm\hat{ }2 for a male. In some embodiments, the composition can be packaged with instructions indicating the amount of the composition that should be applied to the human scalp.

Because of the natural approach to regrowing hair, which involves stimulating blood flow to the scalp, increasing the availability of nutrients to hair follicles, and allowing natural hair regrowth, it is anticipated that hair regrowth for an individual will occur at a rate approximating the rate at which the individual previously lost hair. Thus, if hair was gradually lost over several years, it is expected that hair regrowth will also occur over several years. As another example, it is generally understood that newborn babies take time to grow a full head of hair. It is expected that an individual will regrow hair at a rate somewhat approximating the rate at which a newborn baby first grows hair. In other words, it is not unexpected for the re-growth of hair to take 2 to 3 years, but progress is expected to be noticeable within about 12 months.

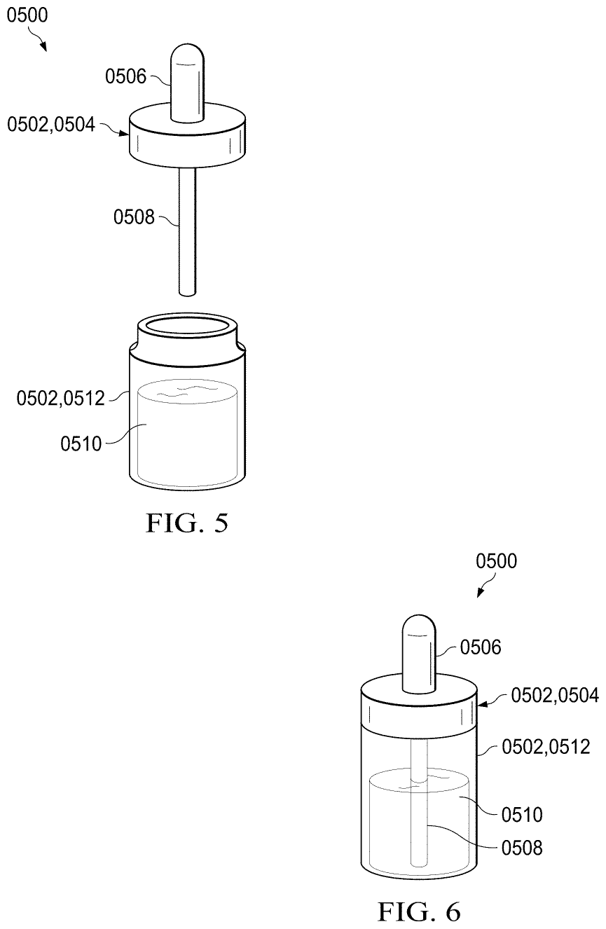

As illustrated in FIG. 5 and FIG. 6, in some embodiments, a system 0500 comprises the composition 0510 of clause 1 and a container 0502. The composition 0510 can be located inside the container 0502 and contained by the container 0502. For example, the container 0502 can comprise a body 0512 and a removable lid 0504, which can assist in containing the composition 0510. FIG. 5 shows an example of a lid 0504 separated (e.g., removed) from the body 0512 of a container 0502. FIG. 6 shows an example of a lid 0504 secured to the body 0512 of the container 0502.

The lid 0504 can be separated from the body 0512, reattached to the body 0512, or any combination thereof using a coupling mechanism, for example, a friction fit between the lid 0504 and the body 0512 or mating screw threads on the lid 0504 and the body 0512, or any coupling mechanism that is known and used for securing a lid 0504 to the body 0512 of a container 0502.

In some embodiments, the container 0502 comprises a dispenser 0506, 0508 for the composition 0510. In some embodiments, the container 0502 can comprise a removable lid 0504, and the lid 0504 can comprise (or be) a dispenser. For example, the dispenser can be in the form of a dropper pipette 0506, 0508. The dropper pipette 0506, 0508 can comprise a conduit 0508 and an elastic bulb 0506 fixed to the conduit 0508. The elastic bulb 0506 can be configured to return to an expanded configuration after being squeezed, thereby creating a vacuum inside the conduit 0508 that can be used to draw a fluid (e.g., liquid, the composition 0510 or any combination thereof) into the conduit 0508. For example, the dropper pipette 0506, 0508 can be configured so that the following ordered sequence of steps can be used to load the conduit 0508 with the composition 0510: (i) the bulb 0506 is squeezed, (ii) the conduit 0508 is inserted into the composition 0510 (e.g., the liquid composition 0510) in the container 0502 (e.g., the body 0512 of the container 0502), (iii) the bulb 0506 is released and the elastic bulb 0506 expands as the composition 0510 is drawn into the conduit 0508, and optionally the dropper pipette 0506, 0508 is configured so that after the conduit 0508 is loaded with the composition 0510, then the composition 0510 can be expelled from the conduit 0508 by squeezing the bulb 0506.

Examples of the composition of carrier oils and essential oils will now be illustrated in the following Tables. As can be seen, several of the tables list a measured value or specific value (“value”) for a parameter as well as a “minimum value” and “maximum value” for the parameter. For each of the following Tables, unless otherwise specified: (1) in some embodiments the “%” reported for a component of an oil can mean “weight %,” and the weight % of each listed component in the table is reported as a percentage of the total weight of the oil described in the table, and (2) in some embodiments the “%” reported for a component of an oil can mean “volume %,” and the volume % of each listed component in the table is reported as a percentage of the total weight of the oil described in the table.

TABLE 2
lemongrass oil

| method of | steam distillation of | | | |
|---|---|---|---|---|
| production | Cymbopogon flexuosus | | | |
| physical property | unit | value | minimum | maximum |
| specific gravity | unitless | 0.891 | 0.852 | 0.905 |
| at 20° C. | | | | |
| refractive index | unitless | 1.485 | 1.483 | 1.489 |
| at 20° C. | | | | |
| optical rotation | ° | −2.1 | −4 | 1 |
| at 20° C. | | | | |
| component | unit | value | | |
| Citral-A (Neral) | % | 31.2 | | |
| Citral-B (Geranial) | % | 40.4 | | |
| Citral-A + Citral-B | % | 71.6 | | |

TABLE 3
Helichrysum oil

| method of | steam | | | |
|---|---|---|---|---|
| production | distillation of the flower of | | | |
| | Helichrysum italicum | | | |
| physical property | unit | value | minimum | maximum |
| flash point | ° C. | 53 | | |
| specific gravity at | | | 0.870 | 0.920 |
| 20° C. | | | | |
| component | unit | value | minimum | maximum |
| α-pinene | % | 27.8577 | 20 | 22 |
| limonene | % | 3.1715 | 2.2 | 2.6 or 4 |
| neryl acetate | % | 4.3634 | 4 or 5 | 6 |
| α-copaene | % | 2.0421 | 1 | 3 |
| italicene | % | 2.9889 | 2.5 | 4 |
| italicenes | % | | 2.5 | 4 |
| β-caryophyllene | % | 4.8521 | 4.5 | 5.5 |
| γ-curcumene | % | 19.4967 | 16 | 19 or 20 |
| AR curcumene | % | 2.1582 | 1 | 4 |
| β-selinene | % | 8.5247 | 7 | 10 |
| α-selinene | % | 4.505 | 3 | 5 |
| camphene | % | | 0.3 | 0.5 |
| β-pinene | % | | 0.3 | 0.5 |
| β-mircene | % | | 0.05 | 0.08 |
| α-terpinene | % | | 0.1 | 0.2 |
| p-cimene | % | | 0.05 | 0.15 |
| 1,8 cineole | % | | 0.2 | 0.4 |
| izobutil angelate | % | | 0.1 | 0.3 |
| γ-terpinene | % | | 0.2 | 0.4 |
| α-terpinolene | % | | 0.1 | 0.2 |
| 2 nanonene | % | | 0.4 | 0.6 |
| methyl butyrate | % | | 0.1 | 0.2 |
| trans pinocarveol | % | | 0.01 | 0.09 |
| mt = 170, dione | % | | 0.5 | 0.9 |
| borneole | % | | 0.01 | 0.09 |
| terpinene 4-ol | % | | 0.1 | 0.2 |
| 3,4-octadione | % | | 0.1 | 0.3 |
| α-terpineole | % | | 0.1 | 0.3 |
| nerole | % | | 0.1 | 0.8 |
| 3,5 heptadione | % | | 0.1 | 0.2 |
| mt = 184, dione | % | | 0.01 | 0.1 |
| α-ylangene | % | | 0.1 | 0.4 |
| bergamotene | % | | 0.1 | 1 |
| trans a | | | | |
| mt = 210, italdione | % | | 1 | 4 |
| neryl proplonate | % | | 0.5 | 1 |
| γ-selinene | % | | 0.1 | 0.2 |
| linalool | % | | 0.02 | 0.04 |
| bergamotene cis α | % | | 0.8 | 1 |
| mt = 224, dione | % | | 1 | 3 |
| [symbol] Cadinene | % | | 1 | 3 |

TABLE 4
Lavender Oil

| method of | steam distillation of the flowering tops of | | | |
|---|---|---|---|---|
| production | Lavandula angustifolia | | | |
| physical | | | | |
| property | unit | value | minimum | maximum |
| specific gravity | unitless | | 0.8750 | 0.8980 |
| at 20° C. | | | | |
| refractive index | unitless | | 1.4570 | 1.4700 |
| at 20° C. | | | | |
| boiling point | ° C. | 204 | | |
| flash point | ° C. | 72 | | |
| solubility in | g/L | 1.15 | | |
| water at 20° C. | | | | |
| solubility in 75% | | 1:2 | | |
| ethanol | | | | |
| component | unit | value | minimum | maximum |
| 1,8-cineol | % | 0.61 | 0 | 1.5 |
| cis-β-ocimene | % | 5.89 | 3 | 9.5 |
| trans-β- | % | 2.99 | 1 | 6 |
| ocimene | | | | |
| camphor | % | 0.2 | 0 | 0.5 |
| linalool | % | 35.72 | 20 | 37 |
| linalyl acetate | % | 26.36 | 27 | 55 |
| 1-terpinene-4-ol | % | 5.14 | 1 | 4 |
| lavandulyl | % | 4.13 | 0 | 2 |
| acetate | | | | |
| lavandulol | % | 0.86 | 0.5 | 3 |
| limonene | % | | 0 | 1 |
| α terpineol | % | | 0 | 1 |

TABLE 5
Clary Sage Oil

| method of | Steam distillation of the flowering tops and leaves | | | |
|---|---|---|---|---|
| production | of Salvia sclarea L. | | | |
| physical | | | | |
| property | unit | value | minimum | maximum |
| specific gravity at | unitless | 0.90 | 0.890 | 0.910 |
| 20° C. | | | | |
| refractive index | unitless | | 1.4550 | 1.4700 |
| at 20° C. | | | | |
| optical rotation | ° | | −25.00 | −6.00 |
| at 20° C. | | | | |
| boiling point | ° C. | 210 | 190 | 230 |
| flash point | ° C. | 75 | 75 | 86.1 |
| | | | | (187° F.) |
| component | unit | value | minimum | maximum |
| Myrcene | % | 0.86 | | |
| Limonene | % | 0.51 | 0 | 1 |
| E-Ocimene | % | 0.35 | | |
| α-copaene | % | 0.75 | | |
| linalool | % | 9.90 | 0 | 25 |
| linalyl-acetate | % | 72.20 | 50 | 80 |
| β-caryophyllene | % | 2.11 | 0 | 3 |
| germacrene D | % | 2.57 | 0 | 4 |
| α-terpineol | % | 1.38 | 0 | 3 |
| neryl acetate | % | 1.17 | 0 | 1.7 |
| alkene | % | 0.34 | | |
| geranyl acetate | % | 2.06 | 2 | 3 |
| Nerol | % | 0.52 | | |
| Geraniol | % | 1.33 | 0 | 2 |
| caryophyllene | % | 1.17 | 0 | 0.5 |
| oxide | | | | |
| spathulenol | % | 0.13 | | |
| alcohol form of | % | 0.68 | 0.5 | 3.0 |
| listed esters | | | | |

TABLE 6
Cedarwood Oil

| method of | steam-distillation from the wood of Juniperus | | | |
|---|---|---|---|---|
| production | Virginiana L. | | | |
| physical | | | | |
| property | unit | value | minimum | maximum |
| specific gravity | unitless | 0.9556 | 0.9380 | 0.9620 |
| at 25° C. | | | | |
| refractive index | unitless | 1.5056 | 1.5010 | 1.5100 |
| at 20° C. | | | | |
| optical rotation | ° | −5.0 | −36.0 | −16.0 |
| at 20° C. | | | | |
| boiling range | ° C. | 150 | 300 | |
| (initial boiling | | | | |
| point is 150° C.) | | | | |
| flash point | ° C. | 100 | 100 | 113 |
| component | unit | value | minimum | maximum |
| thujopsene | % | 27.45 | 15 | 32 |
| cedrol | % | 21.52 | 14 | 24 |

TABLE 7
Eucalyptus oil

| method of | steam distillation of the leaves of a plant of the | | | |
|---|---|---|---|---|
| production | Eucalyptus genus (e.g., Eucalyptus globulus) | | | |
| physical | | | | |
| property | unit | value | minimum | maximum |
| specific gravity | unitless | 0.9165 | 0.915 | 0.917 |
| at 20° C. | | | | |
| refractive index | unitless | 1.4632 | | |
| at 20° C. | | | | |
| optical rotation | ° C. | +1.75 | | |
| at 20° C. | | | | |
| vapor pressure | mm Hg | 1.7 | | |
| at 20° C. | | | | |
| flash point | ° C. | 42.7778 | | |
| component | unit | value | minimum | maximum |
| cineole | % | 69.041 | | |

TABLE 8
Thyme Oil

| method of | Steam distillation of the whole plant (optionally | | | |
|---|---|---|---|---|
| production | partially dried) Thymus vulgaris L., Lamiaceae or | | | |
| | Thymus zygis L. | | | |
| physical | | | | |
| property | unit | value | minimum | maximum |
| specific gravity | unitless | 0.917 | | |
| at 25° C. | | | | |
| refractive index | unitless | 1.502 | | |
| at 20° C. for the | | | | |
| D line (n20/D) | | | | |
| boiling point | ° C. | 195 | | |
| flash point | ° C. | 62.8 | | |
| | | (145° F.) | | |
| component | unit | value | minimum | maximum |
| ALPHA PINENE | % | | 5 | 10 |
| CARVACROL | % | | 1 | 5 |
| LIMONENE | % | | 1 | 5 |
| LINALOOL | % | | 5 | 10 |
| THYMOL | % | | 30 | 60 |

TABLE 9
Bergamot oil

| method of | extracted by cold press from the outer part of fresh bergamot peel (e.g., | | | | | |
|---|---|---|---|---|---|---|
| production | the peel of the fruit of Citrus bergamia) | | | | | |
| physical | | | | | | |
| property | unit | value | minimum | | maximum | |
| specific gravity | unitless | 0.8750 | 0.8750 | | 0.8840 | |
| at 20° C. | | | | | | |
| refractive index | unitless | 1.4680 | 1.4640 | | 1.4690 | |
| at 20° C. | | | | | | |
| optical rotation | ° | +27.00 | +15 | | +34 | |
| at 20° C. | | | | | | |
| boiling point | ° C. | 180 | | | | |
| flash point | ° C. | 58 | | | | |
| kinematic | mm\hat{ }2/s | | more than | | 7 | |
| viscosity at 40° C. | | | 0 | | | |
| | | | | | alternate | alternate |
| component | unit | value | minimum | maximum | minimum | maximum |
| (R)-P-MENTHA- | % | | 30 | 45 | 30 | 45 |
| 1,8-DIENE | | | | | | |
| LINALYL | % | | 22 | 36 | 22 | 36 |
| ACETATE | | | | | | |
| B-PINENE | % | | 5.5 | 9.5 | 5.5 | 9.5 |
| LINALOOL | % | | 3 | 16 | 3 | 16 |
| GAMMA | % | | 5 | 12 | 5 | 12 |
| TERPINENE | | | | | | |
| ALPHA PINENE | % | | 0.7 | 1.8 | 0 | 2.5 |

TABLE 10
Tea Tree Oil

| method of | steam distillation of the Melaleuca alternifolia | | | |
|---|---|---|---|---|
| production | plant (e.g., leaves and/or terminal branches) | | | |
| physical | | | | |
| property | unit | value | minimum | maximum |
| refractive index | unitless | 1.4780 | 1.475 | 1.482 |
| at 20° C. | | | | |
| optical rotation | ° | +9.53 | +5 | +15 |
| at 20° C. | | | | |
| boiling point | ° C. | 176 | 115 | 270 |
| flash point | ° C. | | 57 | 60 |
| ignition | ° C. | 72 | | |
| temperature | | | | |
| specific gravity | | 0.886 | 0.885 | 0.906 or |
| @ 20° C. | | | | 0.915 |
| component | unit | value | minimum | maximum |
| α-pinene | % | 2.412 | | |
| sabinene | % | 1.058 | | |
| α-terpinene | % | 11.120 | | |
| limonene | % | 1.061 | | |
| p-cymene | % | 1.042 | | |
| 1,8-cineole | % | 3.809 | | |
| γ-terpinene | % | 21.460 | | |
| terpinolene | % | 3.791 | | |
| terpinen-4-ol | % | 39.551 | | |
| α-terpineol | % | 2.676 | | |

TABLE 11
Rosemary oil

| method of | steam distillation of the flowering tops of the | | | |
|---|---|---|---|---|
| production | rosemary plant (i.e., Rosmarinus officinalis) | | | |
| physical | | | | |
| property | unit | value | minimum | maximum |
| specific gravity | unitless | 0.908 | 0.880 | 0.924 |
| at 20° C. | | | | |
| refractive index | unitless | 1.463 | 1.450 | 1.480 |
| at 20° C. | | | | |
| optical rotation | ° | +2.98 | −5 | +5 |
| at 20° C. | | | | |
| flash point | ° C. | 50 | | |
| | | (122° F.) | | |
| component | unit | value | minimum | maximum |
| bornan-2-on | % | | 10 | 19.99 |
| α-pinene | % | | 5 | 9.99 |
| β-pinene | % | | 5 | 9.99 |
| limonene | % | | 2.5 | 5 |
| camphene | % | | 2.5 | 5 |
| β-caryophyllene | % | | 1 | 2.49 |
| p-menth-1-en-8- | % | | 1 | 2.49 |
| ol | | | | |
| myrcene | % | | 1 | 2.49 |
| p-Mentha-1,4- | % | | 1 | 2.49 |
| dien | | | | |
| p-cymene | % | | 1 | 2.49 |
| terpinolene | % | | 0 | <1 |

TABLE 12
Peppermint Oil

| method of | steam distillation of the fresh or partly | | | |
|---|---|---|---|---|
| production | dried Mentha piperita plant, above ground, just | | | |
| | before flowering | | | |
| physical | | | | |
| property | unit | value | minimum | maximum |
| specific gravity | unitless | 0.90 | | |
| at 20° C. | | | | |
| refractive index | unitless | 1.461 | | |
| at 20° C. | | | | |
| flash point | ° C. | 62.8 | | |
| component | unit | value | minimum | maximum |
| L-menthol | % | 32.890 | 30 | 40 |
| Menthafuron | % | 4.267 | 2.5 | 5.5 |

TABLE 13
Coconut Oil (e.g., Extra Virgin Coconut Oil)

| physical | | | | |
|---|---|---|---|---|
| property | unit | value | minimum | maximum |
| peroxide value | meq O₂/kg | 0.15 | | |
| moisture | % | 0.05 | | |
| saponification value | mg KOH/g | 251.3 | | |
| Standard plate count | CFU/g | <10 | 0 | 1500 |
| mold | CFU/g | <10 | 0 | 100 |
| free fatty acid (% Lauric) | % | 0.02 | | |
| fatty acid component | | | | |
| (wt. % or vol. %) or fatty | | | | |
| acid profile (Area %) | unit | value | minimum | maximum |
| c:8:0 Caprylic | % | 7.3 | 5 | 10 |
| c:10:0 Capric | % | 5.6 | 4 | 9.7 |
| c:12:0 Lauric | % | 53.2 | 44 | 55 |
| c:14:0 Myristic | % | 19.7 | 13 | 21 |
| c:16:0 Palmtic | % | 7.5 | 6 | 11 |
| c:18:0 Linoleic | % | 0.4 | 0.25 | 4 |

TABLE 14
Castor Oil

| method | extracted by pressing (e.g., cold-pressing) and/or solvent (e.g., n- | | | |
|---|---|---|---|---|
| of production | hexane) from the seed of Ricinus communis | | | |
| physical | | | | |
| property | unit | value | minimum | maximum |
| refractive index | unitless | | 1.4758 | 1.4798 |
| at 20° C. | | | | |
| for the D line (n20/D) | | | | |
| optical rotation | ° | | +3.5 | +6 |
| at 20° C. | | | | |
| boiling point | ° C. | 313 | | |
| | | (595° F.) | | |
| flash point | ° C. | 229 | | |
| | | (445° F.) | | |
| specific gravity | unitless | 0.945 | 0.965 | |
| at 20° C. | | | | |
| viscosity | mPa * s | | 950 | 1050 |
| at 20° C. | | | | |
| Acid Value | mg KOH/g | 0.32 | 0 or greater than 0 | 3 |
| Free Fatty Acid Value | % | 0.16 | 0 or greater than 0 | 1.5 |
| Saponification Value | % | 181.01 | 175 | 185 |
| Unsaponifiable Matter | % | 0.34 | 0 or greater than 0 | 0.8 |
| Hydroxyl Value | mg | 166.06 | 150 | 168 |
| Iodine Value | g l2/100 g | 83.89 | 82 | 90 |
| Specific Gravity | unitless | 0.9568 | 0.954 | 0.980 |
| at 30° C. | | | | |
| | | Value | | |
| | | (example | | |
| | | representative | | |
| fatty acid | | of oil from | | |
| component | unit | India) | minimum | maximum |
| Ricinoleic (C18:1-OH) | % | 87.3 | 75.77 | 94.59 |
| Oleic (C18:1) | % | 4.69 | 2.05 | 7.4 |
| Linoleic (C18:2) | % | 4.92 | 0 or not detected | 8.94 |
| Stearic (C18:0) | % | 1.241 | 0 or not detected | 3.05 |
| Palmitic (C16:1) | % | 1.016 | 0.31 | 2.77 |
| Linolenic (C18:3) | % | 0.63 | 0 or not detected | 0.63 |

TABLE 15
Grape Seed Oil (e.g., Virgin Grape Seed Oil)

| physical | | | | |
|---|---|---|---|---|
| property | unit | value | minimum | maximum |
| Free fatty acid | % | 0.7 | | |
| (FFA) | | | | |
| peroxide value | meq O₂/kg | | 20 | 4.45 |
| fatty acid | | | | |
| component (wt. | | | | |
| % or vol. %) or | | | | |
| fatty acid | | | | |
| profile (area %) | unit | Value | minimum | maximum |
| c:16 Palmitic | % | 9.26 | 6 | 10 |
| c:18 Stearic | % | 4.61 | 3.0 | 6 |
| c:18:1 Oleic | % | 18.42 | 12 | 28 |
| c:18:2 Linoleic | % | 66.6 | 60 | 76 |
| c:18:3 Linolenic | % | 0.35 | 0 | 0.5 |
| c:20 Arachidic | % | 0.31 | 0 | 0.5 |
| c:20:1 Gadoleic | % | 0.13 | 0 | 2.0 |

TABLE 16
Almond Oil (e.g., Virgin Sweet Almond Oil)

| physical | | | | |
|---|---|---|---|---|
| property | unit | value | minimum | maximum |
| acid value | mg KOH/g | 0.3 | 0 | 3.0 |
| peroxide value | meq O₂/kg | 3.3 | 0 | 15.0 |
| fatty acid | | | | |
| component (wt. | | | | |
| % or vol. %) or | | | | |
| fatty acid | | | | |
| profile (area %) | unit | value | minimum | maximum |
| c:16:0 Palmitic | % | 7.4 | | |
| c:18:0 Stearic | % | 2.40 | | |
| c:18:1 Oleic | % | 63.9 | | |
| c:18:2 Linoleic | % | 21.7 | | |

TABLE 17
Avocado Oil

| physical | | | | |
|---|---|---|---|---|
| property | unit | value | minimum | maximum |
| acid value | mg KOH/g | 0.63 | | 10 |
| peroxide value | meq O₂/kg | 4.40 | | 20 |
| fatty acid | | | | |
| component (wt. | | | | |
| % or vol. %) or | | | | |
| fatty acid profile | | | | |
| (area %) | unit | value | minimum | maximum |
| c:16:0 Palmitic | % | 13 | 12 | 25 |
| c:16:1 Palmitoleic | % | 4.2 | 2 | 12 |
| c:18:1 Oleic | % | 70.8 | 45 | 75 |
| c:18:2 Linoleic | % | 7.0 | 5 | 16 |

TABLE 18
Jojoba Oil

| physical | | | | |
|---|---|---|---|---|
| property | unit | value | minimum | maximum |
| iodine value | g l2/100 g | 81.64 | 80 | 92 |
| acid value | mg KOH/g | 0.18 | 0 | 2 |
| peroxide value | meq O₂/kg | 0.7 | 0 | 10.0 |
| saponification | mg KOH/g | 97.2 | 85.0 | 98.0 |
| value | | | | |
| Specific Gravity | | 0.860 | 0.860 | 0.873 |
| at 25° C. | | | | |

TABLE 19
Apricot Oil (e.g., Apricot Kernel Oil)

| physical | | | | |
|---|---|---|---|---|
| property | Unit | Value | minimum | maximum |
| acid value | mg KOH/g | <1.5 | 0 | 10 |
| peroxide value | meq O₂/kg | <0.8 | 0 | 10 |
| fatty acid | | | | |
| component | unit | Value | minimum | maximum |
| c:16:0 Palmitic | % | 5.3 | 3 | 7.5 |
| c:16:1 Palmitoleic | % | 0.8 | 0 | 2 |
| c:18:0 Stearic | % | 1.3 | 0 | 3 |
| c:18:1 Oleic | % | 66.5 | 62 | 76 |
| c:18:2 Linoleic | % | 25.8 | 19 | 33 |

### Additional Embodiments

The following clauses are offered as further description of the disclosed invention:

1. A composition (e.g., to enhance the appearance of human hair, to enhance the health of human hair, to restore human hair, to increase the thickness of human hair (e.g., to increase the thickness of an individual hair strand), to increase the fullness of human hair (e.g., to increase the thickness of individual hair strands over an area of the scalp), to increase the size of human hair follicles, to increase the size of human hair roots, to enhance the growth of human hair, to mitigate the loss of human hair, to stop or reverse human hair loss, or any combination thereof, optionally wherein the hair is on the head (e.g., the scalp) of a human, optionally wherein the human is a male, a female or either a male or female), the composition comprising:

essential oil, the essential oil comprising: lemongrass oil, helichrysum oil, lavender oil, clary sage oil, cedarwood oil, eucalyptus oil, thyme oil, bergamot oil, tea tree oil, rosemary oil, peppermint oil, or any combination thereof; and

carrier oil (e.g., capable of carrying the essential oil, capable of forming a homogenous mixture with the essential oil, miscible with the essential oil in any mass ratio or volume ratio without separation of the essential oil and the carrier oil, or any combination thereof);

optionally: the carrier oil comprising at least two oils from mutually distinct plant species, at least three oils from mutually distinct plant species, coconut oil, castor oil, apricot kernel oil, grape seed oil, almond oil (e.g., sweet almond oil), avocado oil, Jojoba oil (e.g., golden Jojoba oil), any selection of at least 2 of the preceding oils, any selection of at least 3 of the preceding oils, or any combination thereof;

optionally, the composition comprising: 97-99 vol. % (or mass %) carrier oil and 3-1 vol. % (or mass %) essential oil;

optionally the composition comprises components in the following volume percentages (or mass percentages): 16.34% extra virgin coconut oil, 16.34% castor oil, 16.34% grapeseed oil, 16.34% sweet almond oil, 16.34% virgin avocado oil, 16.34% Jojoba oil (e.g., golden Jojoba oil), 0.18% lemongrass oil, 0.18% helichrysum oil, 0.18% lavender oil, 0.18% clary sage oil, 0.18% cedarwood oil, 0.18% eucalyptus oil, 0.18% thyme oil, 0.18% bergamot oil, 0.18% tea tree oil, 0.18% rosemary oil, 0.18% peppermint oil; optionally wherein the volume percentage (or mass percentage) of each of the components or any combination of the components can vary by +/−300, 200, 100, 90, 80, 70, 66, 60, 50, 40, 30, 20, 10, 9, 8, 7, 6, 5, 4, 3, 2 or 1% from the listed value;

optionally the composition comprises components in the following volume percentages (or mass percentages): 16.34% extra virgin coconut oil, 16.34% apricot oil, 16.34% grapeseed oil, 16.34% sweet almond oil, 16.34% virgin avocado oil, 16.34% Jojoba oil (e.g., golden Jojoba oil), 0.18% lemongrass oil, 0.18% helichrysum oil, 0.18% lavender oil, 0.18% clary sage oil, 0.18% cedarwood oil, 0.18% eucalyptus oil, 0.18% thyme oil, 0.18% bergamot oil, 0.18% tea tree oil, 0.18% rosemary oil, 0.18% peppermint oil; optionally wherein the volume percentage (or mass percentage) of each of the components or any combination of the components can vary by +/−300, 200, 100, 90, 80, 70, 66, 60, 50, 40, 30, 20, 10, 9, 8, 7, 6, 5, 4, 3, 2 or 1% from the listed value;

optionally the composition comprises each of the listed component oils of the carrier oil at a volume percentage (or mass percentage) equal to at least ¼, ⅓, ½, ⅔, ¾, ⅘, 9/10, 95/100, 96/100, 97/100, 98/100, or 99/100 and up to 1 times the listed component oil of the carrier oil making up the greatest volume percentage (or mass percentage) of the composition (optionally, there are a plurality of carrier oils that are the carrier oil making up the greatest volume percentage (or mass percentage) of the composition because they are present in the composition at the some volume (or mass));

optionally, the composition comprising at least 13, 14, 15, 16, 17, 18, 19 or 20 vol. % (or mass %), no more than 13, 14, 15, 16, 17, 18, 19 or 20 vol. % (or mass %), or any combination thereof of each of the following components: coconut oil (e.g., extra virgin coconut oil), castor oil, grapeseed oil, sweet almond oil, avocado oil (e.g., virgin avocado oil), and Jojoba oil (e.g., golden Jojoba oil);

optionally, the composition comprising at least 13, 14, 15, 16, 17, 18, 19 or 20 vol. % (or mass %), no more than 13, 14, 15, 16, 17, 18, 19 or 20 vol. % (or mass %), or any combination thereof of each of the following components: coconut oil (e.g., extra virgin coconut oil), apricot oil, grapeseed oil, sweet almond oil, avocado oil (e.g., virgin avocado oil), and Jojoba oil (e.g., golden Jojoba oil);

optionally, the composition comprising at least 13, 14, 15, 16, 17, 18, 19 or 20 vol. % (or mass %), no more than 13, 14, 15, 16, 17, 18, 19 or 20 vol. % (or mass %), or any combination thereof of any selection or any combination of the following components: coconut oil (e.g., extra virgin coconut oil), castor oil, apricot oil, grapeseed oil, sweet almond oil, avocado oil (e.g., virgin avocado oil), and Jojoba oil (e.g., golden Jojoba oil);

optionally the carrier oil comprising the extra virgin coconut oil, the castor oil, the grapeseed oil, the sweet almond oil, the virgin avocado oil, and the Jojoba oil (e.g., golden Jojoba oil);

optionally the carrier oil comprising the extra virgin coconut oil, the apricot oil, the grapeseed oil, the sweet almond oil, the virgin avocado oil, and the Jojoba oil (e.g., golden Jojoba oil);

optionally wherein the ratio of the volume (or mass) of any one listed component of the carrier oil (i.e., the volume (or mass) of the listed component added to other carrier oil components to form the carrier oil) divided by the volume (or mass) of any second listed component of the carrier oil (i.e., the volume (or mass) of the listed second component added to other carrier oil components to form the carrier oil) is at least 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3, no more than 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3, or any combination thereof;

optionally: the carrier oil makes up at least 50, 55, 60, 65, 70, 75, 80, 85, 90, 95, 96, 97, 97.25, 97.5, 97.75, 98, 98.25, 98.5, 98.75 or 99 vol. % (or wt. %) of the composition, the carrier oil makes up no more than 50, 55, 60, 65, 70, 75, 80, 85, 90, 95, 96, 97, 97.25, 97.5, 97.75, 98, 98.25, 98.5, 98.75 or 99 vol. % (or wt. %) of the composition, or any combination thereof;

optionally the composition comprises each of the listed component oils of the essential oil at a volume percentage (or mass percentage) equal to at least ¼, ⅓, ½, ⅔, ¾, ⅘, 9/10, 95/100, 96/100, 97/100, 98/100, or 99/100 and up to 1 times the listed component oil of the essential oil making up the greatest volume percentage (or mass percentage) of the composition (optionally, there are a plurality of essential oils that are the essential oil making up the greatest volume percentage (or mass percentage) of the composition because they are present in the composition at the some volume (or mass));

optionally the composition comprising at least 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.46, 0.47, 0.48, 0.49, 0.5, 0.51, 0.52, 0.53, or 0.54 vol. % (or mass %), no more than 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.46, 0.47, 0.48, 0.49, 0.5, 0.51, 0.52, 0.53, 0.54, or 0.55 vol. % (or mass %), or any combination thereof of each of the following components: lemongrass oil, helichrysum oil, lavender oil, clary sage oil, cedar wood oil, eucalyptus oil, thyme oil, bergamot oil, tea tree oil, rosemary oil, and peppermint oil;

optionally the essential oil comprising: the lemongrass oil, the helichrysum oil, the lavender oil, the clary sage oil, the cedar wood oil, the eucalyptus oil, the thyme oil, the bergamot oil, the tea tree oil, the rosemary oil, and the peppermint oil;

optionally wherein the ratio of the volume (or mass) of any one listed component of the essential oil (i.e., the volume (or mass) of the listed component added to other essential oil components to form the essential oil) divided by the volume (or mass) of any second listed component of the essential oil (i.e., the volume (or mass) of the listed second component added to other essential oil components to form the essential oil) is at least 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3, no more than 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3, or any combination thereof;

optionally: the essential oil makes up at least 1, 1.25, 1.5, 1.75, 2, 2.1, 2.2, 2.25, 2.3, 2.4, 2.5, 2.75 or 3 wt. % of the composition, the essential oil makes up no more than 1, 1.25, 1.5, 1.75, 2, 2.1, 2.2, 2.25, 2.3, 2.4, 2.5, 2.75 or 3 wt. % of the composition, or any combination thereof;

optionally: any component oil of the essential oil is present in the essential oil at a mass fraction equal to at least 1/(3*N), 1/(2*N), 1/(N), 2/N, or 3/N, equal to no more than 1/(3*N), 1/(2*N), 1/(N), 2/N, or 3/N, or any combination thereof; wherein N is the total number of component oils (e.g., different essential oils) in the essential oil, optionally wherein N is at least 1, 2, 3, 4, 5, 6, 7, 8, 9, 10 or 11, optionally wherein N is no more than 1, 2, 3, 4, 5, 6, 7, 8, 9, 10, 11, 12, 13, 14, or 15 or any combination thereof;

optionally: any component oil of the essential oil (i.e., individually), each component oil in the essential oil (i.e., individually), any combination of the components oils of the essential oil (i.e., collectively), or the combination of all the components oils of the essential oil (i.e., collectively) is present in the composition at a mass fraction (e.g., the mass of the component oil divided by the mass of the composition) or volume fraction (e.g., the volume of the component oil divided by the volume of the composition) equal to at least 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.46, 0.47, 0.48, 0.49, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 vol. % (or wt. %), equal to no more than 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, 0.46, 0.47, 0.48, 0.49, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 vol. % (or wt. %), or any combination thereof; wherein N is the total number of component oils (e.g., different essential oils) in the essential oil;

optionally: the lemongrass oil being from Cymbopogon flexuosus, the lemongrass oil being essential oil extracted by steam distillation of leaves of Cymbopogon flexuosus (e.g., (i) fresh or (ii) partly fresh and partly dried, or (iii) dried Cymbopogon flexuosus), the lemongrass oil comprising or having any characteristic or any combination of characteristics (e.g., method of production, physical property, chemical property, compositional property, mass percentage of a component or molecule, volume percentage of a component or molecule, or any combination thereof) listed in Table 2, or any combination thereof;

optionally: the helichrysum oil being from Hehchrysum italicum, the helichrysum oil being essential oil extracted by steam distillation of flowers of Hehchrysum italicum (e.g., (i) fresh or (ii) partly fresh and partly dried, or (iii) dried Hehchrysum italicum), the helichrysum oil comprising or having any characteristic or any combination of characteristics (e.g., method of production, physical property, chemical property, compositional property, mass percentage of a component or molecule, volume percentage of a component or molecule, or any combination thereof) listed in Table 3, or any combination thereof;

optionally: the lavender oil being from Lavandula angustifolia, the lavender oil being essential oil extracted by steam distillation of flowering tops of Lavandula angustifolia (e.g., (i) fresh or (ii) partly fresh and partly dried, or (iii) dried Lavandula angustifolia), the lavender oil comprising or having any characteristic or any combination of characteristics (e.g., method of production, physical property, chemical property, compositional property, mass percentage of a component or molecule, volume percentage of a component or molecule, or any combination thereof) listed in Table 4, or any combination thereof;

optionally: the clary sage oil being from Salvia sclarea L., the clary sage oil being essential oil extracted by steam distillation of flowering tops, leaves or a combination thereof of Salvia sclarea L. (e.g., (i) fresh or (ii) partly fresh and partly dried, or (iii) dried Salvia sclarea L.), the clary sage oil comprising or having any characteristic or any combination of characteristics (e.g., method of production, physical property, chemical property, compositional property, mass percentage of a component or molecule, volume percentage of a component or molecule, or any combination thereof) listed in Table 5, or any combination thereof;

optionally: the cedarwood oil being from Juniperus virginiana L., the cedarwood oil being essential oil extracted by steam distillation of wood of Juniperus virginiana L. (e.g., (i) fresh or (ii) partly fresh and partly dried, or (iii) dried Juniperus virginiana L.), the cedarwood oil comprising or having any characteristic or any combination of characteristics (e.g., method of production, physical property, chemical property, compositional property, mass percentage of a component or molecule, volume percentage of a component or molecule, or any combination thereof) listed in Table 6, or any combination thereof;

optionally: the eucalyptus oil being from Eucalyptus radiata, the eucalyptus oil being essential oil extracted by steam distillation of leaves of Eucalyptus radiata (e.g., (i) fresh or (ii) partly fresh and partly dried, or (iii) dried Eucalyptus radiata), the eucalyptus oil comprising or having any characteristic or any combination of characteristics (e.g., method of production, physical property, chemical property, compositional property, mass percentage of a component or molecule, volume percentage of a component or molecule, or any combination thereof) listed in Table 7, or any combination thereof;

optionally: the thyme oil being from Thymus vulgaris L., Thymus zygis L., or a combination thereof, the thyme oil being essential oil extracted by steam distillation of the whole plant (optionally while flowering) of Thymus vulgaris L., Thymus zygis L., or a combination thereof, (e.g., (i) fresh or (ii) partly fresh and partly dried, or (iii) dried Thymus vulgaris L.), the thyme oil comprising or having any characteristic or any combination of characteristics (e.g., method of production, physical property, chemical property, compositional property, mass percentage of a component or molecule, volume percentage of a component or molecule, or any combination thereof) listed in Table 8, or any combination thereof;

optionally: the bergamot oil being from Citrus bergamia, the bergamot oil being essential oil extracted by cold press from the outer part of bergamot peel (e.g., (i) fresh or (ii) partly fresh and partly dried, or (iii) dried bergamot peel), the bergamot oil comprising or having any characteristic or any combination of characteristics (e.g., method of production, physical property, chemical property, compositional property, mass percentage of a component or molecule, volume percentage of a component or molecule, or any combination thereof) listed in Table 9, or any combination thereof;

optionally: the tea tree oil being from Melaleuca alternifolia, the tea tree oil being essential oil extracted by steam distillation of leaves, terminal branches, or combination thereof of Melaleuca alternifolia (e.g., (i) fresh or (ii) partly fresh and partly dried, or (iii) dried Melaleuca alternifolia), the tea tree oil comprising or having any characteristic or any combination of characteristics (e.g., method of production, physical property, chemical property, compositional property, mass percentage of a component or molecule, volume percentage of a component or molecule, or any combination thereof) listed in Table 10, or any combination thereof;

optionally: the rosemary oil being from Rosmarinus officinalis, the rosemary oil being essential oil extracted by steam distillation of flowering tops of Rosmarinus officinalis (e.g., (i) fresh or (ii) partly fresh and partly dried, or (iii) dried Rosmarinus officinalis), the rosemary oil comprising or having any characteristic or any combination of characteristics (e.g., method of production, physical property, chemical property, compositional property, mass percentage of a component or molecule, volume percentage of a component or molecule, or any combination thereof) listed in Table 11, or any combination thereof;

optionally: the peppermint oil being from Mentha piperita, the peppermint oil being essential oil extracted by steam distillation of Mentha piperita (e.g., (i) fresh or (ii) partly fresh and partly dried, or (iii) dried Mentha piperita, optionally above ground portions of the Mentha piperita, optionally just before flowering, optionally for each plant or any group of plants: while flower buds are on the plant or a plant in the group of plants but before any of the flower buds open, optionally for each plant or any group of plants: within the 24-hour period after the first flower bud appears on the plant or any plant in the group of plants), the peppermint oil comprising or having any characteristic or any combination of characteristics (e.g., method of production, physical property, chemical property, compositional property, mass percentage of a component or molecule, volume percentage of a component or molecule, or any combination thereof) listed in Table 12, or any combination thereof;

optionally: the coconut oil being from Cocos nucifera, the coconut oil being oil extracted from the kernel or meat of mature coconuts from Cocos nucifera, the coconut oil comprising or having any characteristic or any combination of characteristics (e.g., method of production, physical property, chemical property, compositional property, mass percentage of a component or molecule, volume percentage of a component or molecule, or any combination thereof) listed in Table 13, or any combination thereof;

optionally: the castor oil being from Ricinus communis, the castor oil being oil extracted from seeds of Ricinus communis (e.g., by pressing (e.g., cold-pressing) and/or solvent (e.g., n-hexane) extraction), the castor oil comprising or having any characteristic or any combination of characteristics (e.g., method of production, physical property, chemical property, compositional property, mass percentage of a component or molecule, volume percentage of a component or molecule, or any combination thereof) listed in Table 14, or any combination thereof;

optionally: the grape seed oil being from the genus Vitis (e.g., Vitis vinifera), the grape seed oil being oil extracted from seeds of the genus Vitis (e.g., Vitis vinifera), the grape seed oil comprising or having any characteristic or any combination of characteristics (e.g., method of production, physical property, chemical property, compositional property, mass percentage of a component or molecule, volume percentage of a component or molecule, or any combination thereof) listed in Table 15, or any combination thereof;

optionally: the almond oil being sweet almond oil (e.g., from Prunus dulcis), the almond oil being oil extracted from seeds of Prunus dulcis, the almond oil comprising or having any characteristic or any combination of characteristics (e.g., method of production, physical property, chemical property, compositional property, mass percentage of a component or molecule, volume percentage of a component or molecule, or any combination thereof) listed in Table 16 or any combination thereof;

optionally: the avocado oil being from Persea americana, the avocado oil being oil extracted from fruit pulp of Persea americana (e.g., by pressing the dehydrated (optionally sliced) fruit pulp of Persea americana), the avocado oil comprising or having any characteristic or any combination of characteristics (e.g., method of production, physical property, chemical property, compositional property, mass percentage of a component or molecule, volume percentage of a component or molecule, or any combination thereof) listed in Table 17 or any combination thereof;

optionally: the jojoba oil being from Simmondsia chinensis, the jojoba oil being oil (e.g., in the form of a mixture of liquid wax esters) extracted from seeds of Simmondsia chinensis, the jojoba oil being golden jojoba oil, the jojoba oil being added to the composition as part of a jojoba composition that is at least 95, 96, 97, 98, 99, 99.1, 99.2, 99.3, 99.4, 99.5, 99.6, 99.7, 99.8, 99.9 wt. % and up to 100 wt. % pure jojoba oil, the jojoba oil comprising or having any characteristic or any combination of characteristics (e.g., method of production, physical property, chemical property, compositional property, mass percentage of a component or molecule, volume percentage of a component or molecule, or any combination thereof) listed in Table 18, or any combination thereof;

optionally: the apricot oil being apricot kernel oil, the apricot oil being from Prunus armeniaca, the apricot oil being oil extracted from kernels of Prunus armeniaca (e.g., by pressing kernels of Prunus armeniaca), the apricot oil (e.g., apricot kernel oil) comprising or having any characteristic or any combination of characteristics (e.g., method of production, physical property, chemical property, compositional property, mass percentage of a component or molecule, volume percentage of a component or molecule, or any combination thereof) listed in Table 19 or any combination thereof;

optionally wherein any oil, any combination of oils, or every oil in the essential oil or the carrier oil, or any combination thereof is unrefined oil, oil obtained via mechanical treatment (e.g., mechanical treatment only), oil obtained without chemical treatment, cold-pressed oil, oil obtained via a process that does not increase the temperature of the oil above 120° F., extra virgin oil, oil with no more than 0.8% free acidity, virgin oil, oil with no more than 1.5% free acidity, organic oil (e.g., oil certified in compliance with the terms of the US-Canada Organic Equivalency Arrangement in effect on Nov. 1, 2020, oil certified as organic under the United States Department of Agriculture (USDA)'s organic certification program as set out in the United States Organic Foods Production Act of 1990 and National Organic Program Regulations in effect on Jun. 30, 2009, oil produced without the use of sodium nitrate, oil produced without hydroponic or aeroponic production methods, or any combination thereof), or any combination thereof;

optionally wherein additional embodiments can be provided wherein any listed value or any combination of listed values in Tables 1-19 can vary from the respective value or values listed in Table 1-19 by +/−100, 90, 80, 70, 60, 50, 40, 30, 20, 10, 9, 8, 7, 6, 5, 4, 3, 2, 1, 0.9, 0.8, 0.6, 0.5, 0.4, 0.3, 0.2 or 0.1% of the listed value (for example, the value can vary from [the value−50% of the value] to [the value+100% of the value] in some embodiments);

optionally the composition is at least 50, 60, 70, 80, 90, 91, 92, 93, 94, 95, 96, 97, 98, 99, or 100% organic, up to 60, 70, 80, 90, 91, 92, 93, 94, 95, 96, 97, 98, 99, or 100% organic, or any combination thereof; optionally wherein organic means (i) certified in compliance with the terms of the US-Canada Organic Equivalency Arrangement in effect on Nov. 1, 2020, (ii) certified as organic under the United States Department of Agriculture (USDA)'s organic certification program as set out in the United States Organic Foods Production Act of 1990 and National Organic Program Regulations in effect on Jun. 30, 2009, (iii) produced without the use of sodium nitrate, (iv) produced without hydroponic production methods, (v) produced without the use of aeroponic production methods, or (vi) any combination thereof;

or any combination thereof.

2. A method of making a composition (e.g., the composition of clause 1), the method comprising:

providing a carrier oil (e.g., the carrier oil of clause 1); and

adding essential oil (e.g., the essential oil of clause 1) to the carrier oil;

optionally the providing a carrier oil comprises mixing component carrier oils to provide the carrier oil;

optionally the step of mixing component carrier oils comprises mixing each component carrier oil in liquid form;

optionally the step of adding the essential oil to the carrier oil comprises separately adding each component oil of the essential oil to the carrier oil;

optionally the step of adding the essential oil to the carrier oil comprises mixing the carrier oil and each added component oil of the essential oil for 2 to 4 minutes; for at least 0.5, 1, 2, 3 or 4 minutes; no more than 1, 2, 3, or 4 minutes; or any combination thereof before adding a subsequent component oil of the essential oil to the carrier oil;

optionally the step of adding the essential oil to the carrier oil comprises mixing the carrier oil while each component oil of the essential oil is added to the carrier oil;

optionally the essential oil comprises: (i) a first sub-composition comprising at least one component essential oil and (ii) a second sub-composition comprising at least one component essential oil, the first sub-composition and the second sub-composition being different, and the step of adding the essential oil to the carrier oil comprises adding the second sub-composition to the carrier oil after adding the first sub-composition to the carrier oil, optionally the first sub-composition comprises any one or any combination of the essential oils described in this disclosure; optionally the second sub-composition comprises any one or any combination of the essential oils described in this disclosure;

optionally: the carrier oil comprises coconut oil; the method is configured so that the coconut oil will be liquid when mixed with any other component of the carrier oil or any component of the essential oil; the coconut oil is mixed with any other component of the carrier oil while the coconut oil is liquid (e.g., at a temperature equal to or above the melting point of the coconut oil, for example, 78° F.); the method is configured so that the coconut oil would remain liquid if the coconut were cooled below the melting point of the coconut oil, for example, 78° F., after being mixed with another component of the carrier oil or any component of the essential oil; the method is configured so that the coconut oil remains liquid after the coconut is cooled below the melting point of the coconut oil, for example, 78° F., after being mixed with another component of the carrier oil or any component of the essential oil; or any combination thereof;

optionally the step of adding the essential oil to the carrier oil comprises adding the following components in the sequential order listed: the lemongrass oil, the helichrysum oil, the lavender oil, the clary sage oil, the cedar wood oil, the eucalyptus oil, the thyme oil, the bergamot oil, the tea tree oil, the rosemary oil, and the peppermint oil; optionally each component of the essential oil is added to the carrier oil while the carrier oil and any previously added components are stirred with a stirrer (e.g., via an overhead stirring rod) at a speed (e.g., at least 500, 600, 700, 800, 900, 1000, 1100, 1200, 1300, 1400, 1500, 1600, 1700, 1800, 1900 or 2000 rpm; no more than 600, 700, 800, 900, 1000, 1100, 1200, 1300, 1400, 1500, 1600, 1700, 1800, 1900, 2000 or 2100 rpm; or any combination thereof) and for a time (e.g., 2-4 minutes or 3 minutes; or at least 5, 10, 20, 30, 40, 50, 60, 90, 120, 150, 180, 210, or 240 seconds; no more than 5, 10, 20, 30, 40, 50, 60, 90, 120, 150, 180, 210, or 240 seconds; or any combination thereof) configured to disperse the added essential oil into the carrier oil (e.g., to form a homogeneous mixture) before a subsequent component is added;

or any combination thereof.

3. A method of using a composition (e.g., the composition of clause 1), the method comprising:

applying the composition to a head (e.g., a human head, the entire top of a human head, the head of a human male, a human scalp, the entire scalp, at least 10, 20, 30, 40, 50, 60, 70, 80, 90, 91, 92, 93, 94, 95, 96, 97, 98, 99 or 100% by area of the scalp, no more than 20, 30, 40, 50, 60, 70, 80, 90, 91, 92, 93, 94, 95, 96, 97, 98, 99 or 100% by area of the scalp, hair follicles on the head, the head of a human male, the head of a human female, or any combination thereof);

optionally the step of applying the composition to the head comprises applying 1 to 4 mL of the composition to the head per application of the composition;

optionally the step of applying the composition to the head comprises applying the composition to the head 2 to 4 times per week;

optionally a week is 7 consecutive days;

optionally a day is 24 consecutive hours;

optionally: wherein applying the composition to the head comprises applying the composition to the head (i) 2 to 4 times per week (e.g., at least 2, 3, or 4; no more than 2, 3, or 4 or any combination thereof), wherein a week is 7 consecutive days, and wherein a day is 24 consecutive hours; (ii) on non-consecutive days; (iii) with one or two intervening days or from 1 to 2 intervening non-application days (i.e., days when the composition is not applied to the head) separating consecutive application days (i.e., days when the composition is applied to the head); (iv) every other day or every two days; (v) every three days; (vi) every two or three days; (vii) each application of the composition to the head comprises applying at least 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5 or 4 mL (or mg) per application to the head, applying no more than 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5 or 4 mL (or mg) per application to the head, applying 0.5 to 4 mL (or 0.5 to 4 mg) per application to the head, or any combination thereof; or (viii) any combination thereof;

optionally each application of the composition to the head (and optionally massaging of the composition into the head) occurs within a period of time that is no more than 24, 12, 6, 3, 2, 1, ½, ⅓, ¼, ⅙, 1/12, 1/24, 1/30, or 1/60 hours; at least ½, ⅓, ¼, ⅙, 1/12, 1/24, 1/30, or 1/60 hours; or any combination thereof;

optionally the step of applying the composition to the head comprises applying the composition to the head so that there are 1 to 3 intervening non-application days, at least 0.5, 1, 2, or 3 intervening non-application days, no more than 1, 2, 3, or 4 intervening non-application days, or any combination thereof separating consecutive application days, wherein the intervening non-application days are days when the composition is not applied to the head, and wherein the consecutive application days are days when the composition is applied to the head.

optionally: wherein the method comprises (i) allowing the composition to be absorbed into the head; (ii) allowing the composition to remain on the head for at least 0.5, 1 or 2 hours before any washing of the head; (iii) massaging the composition into the head; or (iv) any combination thereof;

optionally: wherein the amount of the composition applied to the head (e.g., human head, scalp, hair follicles, hair roots or any combination thereof) is at least 1, 1.5, 2, 2.5, 3, 3.5, 4, 4.5 or 5 mL; no more than 1, 1.5, 2, 2.5, 3, 3.5, 4, 4.5 or 5 mL; or any combination thereof;

optionally: wherein the amount of the composition applied to the head (e.g., human head, human scalp, hair follicles, hair roots or any combination thereof) is selected to provide about 1 mL of the composition per at least 55 to 150 cm\hat{ }2 of surface area of scalp to which the composition is applied, or to provide at least 0.0066, 0.0067, 0.0068, 0.0069, 0.007, 0.008, 0.009, 0.01, 0.011, 0.012, 0.013, 0.014, 0.015, 0.016, 0.017 or 0.018 mL composition per cm\hat{ }2 scalp surface area; no more than 0.0066, 0.0067, 0.0068, 0.0069, 0.007, 0.008, 0.009, 0.01, 0.011, 0.012, 0.013, 0.014, 0.015, 0.016, 0.017 or 0.018 mL composition per cm\hat{ }2 scalp surface area; or any combination thereof;

or any combination thereof.

4. A system comprising the composition of clause 1 and a container, the composition located inside the container and contained by the container;

optionally the container comprises a body and a removable lid (e.g., configured to be secured to the body);

optionally the container comprises a dispenser for the composition;

optionally: the container comprises a removable lid, the lid comprises a dispenser, the dispenser is in the form of a dropper pipette, or any combination thereof;

optionally the dropper pipette comprises a conduit and an elastic bulb fixed to the conduit, the elastic bulb configured to return to an expanded configured after being squeezed, thereby creating a vacuum inside the conduit that can be used to draw a fluid (e.g., liquid, the composition or any combination thereof) into the conduit, optionally the dropper pipette is configured so that the following ordered sequence of steps can be used to load the conduit with the composition: (i) the bulb is squeezed, (ii) the conduit is inserted into the composition (e.g., liquid composition) in the container, (iii) the bulb is released and the elastic bulb expands as the composition is drawn into the conduit, and optionally the dropper pipette is configured so that after the conduit is loaded with the composition, then the composition can be expelled from the conduit by squeezing the bulb;

optionally the system comprising instructions for use of the composition;

optionally the instructions being located on the container, located on a sheet that is separate from the container, located on a website (e.g., identified by the container or identified by written information that is separate from the container), or any combination thereof;

optionally the instructions listing, comprising an instruction to perform, providing an instruction to perform, or providing a suggestion to perform at least one or any combination of method steps recited in the method of clause 3;

or any combination thereof.

Although the present disclosure has provided many examples of systems, apparatuses, and methods, it should be understood that the components of the systems, apparatuses and method described herein are compatible and additional embodiments can be created by combining one or more elements from the various embodiments described herein. As an example, in some embodiments, a method described herein can further comprise one or more elements of a system described herein or a selected combination of elements from any combination of the systems or apparatuses described herein.

Furthermore, in some embodiments, a method described herein can further comprise using a system described herein, using one or more elements of a system described herein, or using a selected combination of elements from any combination of the systems described herein.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments. For example, it should be understood that the method steps described herein are exemplary, and upon reading the present disclosure, a skilled person would understand that one or more method steps described herein can be combined, omitted, re-ordered, or substituted in some embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by replacing “comprising” with the phrase “consisting essentially of” or “consisting of.”

Where language, for example, “for” or “to”, is used herein in conjunction with an effect, function, use or purpose, an additional embodiment can be provided by replacing “for,” “to,” “configured for/to,” “formulated for/to,” or “adapted for/to,” with any selection or combination of “for,” “to,” “configured for/to,” “formulated for/to,” or “adapted for/to.”

Additionally, when a range for a particular variable is given for an embodiment, an additional embodiment can be created using a subrange or individual values that are contained within the range. Moreover, when a value, values, a range, or ranges for a particular variable are given for one or more embodiments, an additional embodiment can be created by forming a new range whose endpoints are selected from any expressly listed value, any value between expressly listed values, and any value contained in a listed range. For example, if the application were to disclose an embodiment in which a variable is 1 and a second embodiment in which the variable is 3-5, a third embodiment can be created in which the variable is 1.31-4.23. Similarly, a fourth embodiment can be created in which the variable is 1-5.

As used herein, examples of “substantially” include: “more so than not,” “mostly,” and “at least 30%, 40%, 50%, 60%, 70%, 80%, 90%, 95%, 96%, 97%, 98% or 99%” with respect to a referenced characteristic.

As used herein, examples of “about” and “approximately” include a specified value or characteristic to within plus or minus 30%, 25%, 20%, 15%, 10%, 5%, 4%, 3%, 2%, or 1% of the specified value or characteristic.

Unless otherwise specified, percentages of a component in a composition are given in terms of weight percentages. However, additional embodiments can be formed in which “weight %” or equivalent language is replaced with “volume %.” Similarly, additional embodiments can be formed in which “volume %” or equivalent language is replaced with “weight %.”

When the specific gravity of a substance is referenced in this disclosure, unless otherwise specified, the specific gravity can be defined as the density at 20° C. for the substance divided by the density of water at 4° C. (i.e., 1 g/ml).

Unless otherwise specified, the flash point of a substance can be determined in accordance with any one of ASTM D56-05-16a, ASTM D3278-20, ASTM D3828-16a, ASTM D93-08-20. Accordingly, in some embodiments, the flash point can be deemed to be specified in accordance with one and only one of these test methods. Additionally, for some embodiments, the flash point can be deemed to be obtained by following any selection of or all of these test methods.

Unless otherwise noted the free fatty acid (FFA) content of a sample can be determine in accordance with ASTM D5555-95 (2017).

As used in this disclosure, the “acid value” is the mass in milligrams of potassium hydroxide (KOH) necessary to neutralize one gram of a substance, for example, an oil.

As used in this disclosure, the “saponification value” can be determined using ASTM D5558-95(2017).

As used in this disclosure, the “peroxide value” is the milliequivalents of peroxide oxygen present in a fat or oil per 1 kilogram of the fat or oil. The peroxide value can be determined using IS 3960:2017(en).

As used in this disclosure unsaponifiable matter can be determined in accordance with ASTM D1965-87(1998), which is presently withdrawn.

As used in this disclosure hydroxyl value can be determined in accordance with ASTM D1957-86(2001), which is presently withdrawn.

As used in this disclosure, iodine value is can be determined in accordance with ISO 3961:2018, ASTM D5768-02(2018), ASTM 1959-97 (withdrawn 2006), or ASTM D5554-15.

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

I claim:

1. A composition to increase the thickness of human hair, the composition comprising:
essential oil, and
carrier oil that is miscible with the essential oil in any mass ratio;
the essential oil comprising: lemongrass oil, helichrysum oil, lavender oil, clary sage oil, cedarwood oil, eucalyptus oil, thyme oil , bergamot oil, tea tree oil, rosemary oil, and peppermint oil.

2. The composition of claim 1, wherein the composition comprises 97 to 99 vol. % carrier oil and 3 to 1 vol. % essential oil.

3. The composition of claim 2, wherein the carrier oil comprises at least two oils from mutually distinct plant species.

4. The composition of claim 2, wherein the carrier oil comprises at least three oils from mutually distinct plant species.

5. The composition of claim 2, wherein the carrier oil comprises at least three oils selected from the group consisting of coconut oil, castor oil, apricot kernel oil, grape seed oil, sweet almond oil, avocado oil, and Jojoba oil.

6. The composition of claim 2, wherein the carrier oil comprises coconut oil, apricot kernel oil, grape seed oil, sweet almond oil, avocado oil, and Jojoba oil.

7. The composition of claim 2, wherein the composition comprises at least 0.1% and no more than 0.55% of each of the following components: lemongrass oil, helichrysum oil, lavender oil, clary sage oil, cedar wood oil, eucalyptus oil, thyme oil, bergamot oil, tea tree oil, rosemary oil, and peppermint oil.

8. The composition of claim 2, wherein the composition comprises at least 0.2% and no more than 0.3% of each of the following components: lemongrass oil, helichrysum oil, lavender oil, clary sage oil, cedar wood oil, eucalyptus oil, thyme oil, bergamot oil, tea tree oil, rosemary oil, and peppermint oil.

9. The composition of claim 8, wherein the composition comprises 13 to 20 vol. % of each of the following components: coconut oil, apricot oil, grapeseed oil, sweet almond oil, avocado oil, and Jojoba oil.

10. A method of making the composition of claim 1, the method comprising:
providing the carrier oil; and
adding the essential oil to the carrier oil.

11. The method of claim 10, wherein the step of providing the carrier oil comprises mixing component carrier oils to provide the carrier oil.

12. The method of claim 11, wherein the step of mixing component carrier oils comprises mixing each component carrier oil in liquid form.

13. The method of claim 12, wherein the step of adding the essential oil to the carrier oil comprises separately adding each component oil of the essential oil to the carrier oil.

14. The method of claim 13, wherein the step of adding the essential oil to the carrier oil comprises mixing the carrier oil while each component oil of the essential oil is added to the carrier oil.

15. The method of claim 14, wherein the step of adding the essential oil to the carrier oil comprises mixing the carrier oil and each added component oil of the essential oil for 2 to 4 minutes before adding a subsequent component oil of the essential oil to the carrier oil.

16. A method of using the composition of claim 1, the method comprising applying the composition to a human scalp.

17. The method of claim 16, wherein the step of applying the composition to the head comprises applying 1 to 4 mL of the composition to the head per application of the composition, and wherein the step of applying the composition to the head comprises applying the composition to the head 2 to 4 times per week, wherein a week is 7 consecutive days, and wherein a day is 24 consecutive hours.

18. The method of claim 17, wherein the step of applying the composition to the head comprises applying the composition to the head so that there are 1 to 3 intervening non-application days separating consecutive application days, wherein the intervening non-application days are days when the composition is not applied to the head, and wherein the consecutive application days are days when the composition is applied to the head.

19. The method of claim 18, the method comprising:
allowing the composition to be absorbed into the head.

20. A system comprising the composition of claim 1, a container, and instructions for use of the composition;
the composition located inside the container and contained by the container;
the instructions comprising an instruction to apply the composition to a human scalp.
